(19) United States
(12) Patent Application Publication
(10) Pub. No.: US 2014/0356253 A1
(43) Pub. Date: Dec. 4, 2014

(54) BIOMEDICAL DEVICES

(71) Applicant: ETERNAL CHEMICAL CO., LTD., Kaohsiung (TW)

(72) Inventors: Tu-Yi Wu, KAOHSIUNG (TW); Chao-Min Cheng, KAOHSIUNG (TW); Hsi-Kai Wang, KAOHSIUNG (TW); Yen-Ting Yeh, KAOHSIUNG (TW); Wei-Ming Tu, KAOHSIUNG (TW)

(73) Assignee: ETERNAL CHEMICAL CO., LTD., KAOHSIUNG (TW)

(21) Appl. No.: 14/287,649

(22) Filed: May 27, 2014

Related U.S. Application Data
(60) Provisional application No. 61/827,862, filed on May 28, 2013.

(51) Int. Cl. G01N 33/52; G01N 31/22

(52) U.S. Cl. CPC G01N 33/525; G01N 31/227
USPC 422/423; 29/458

(57) ABSTRACT

The present invention provides a biomedical device comprising a porous hydrophilic substrate; a hydrophobic material; a fluid inlet; two or more test zones, wherein fluid vertically flows through the porous hydrophilic substrate and then distributes into the test zones. The present invention further provides a method for making a biomedical device.

### CROSS REFERENCE APPLICATION

This application claims the benefit of U.S. Provisional Application No. 61/827,862, filed May 28, 2013, the entire contents of which are hereby incorporated by reference, as if fully contained herein.

### FIELD OF THE INVENTION

The present invention is in the technical field of biomedical devices. In particular, the present invention is directed to biomedical devices with a porous substrate, methods for making the same, and methods of using the same.

### BACKGROUND OF THE INVENTION

In view of the inconvenience and difficulty in implementing conventional biological fluid measurements in geographically remote regions and the costs associated with clinical analysis, there is need for an easy-to-make, easy-to-handle and robust biomedical device. For instance, glucose meters for home blood glucose monitoring have been available on the market for years. However, the high price of these devices and the accompanying test sample holders make them unappealing to consumers. In addition to the disadvantages related to cost and accessibility, the current commercially available home biological fluid measurement devices, which can test only one sample input at a time are also criticized for ineffective utilization of biological fluid. To improve the efficiency of biological measurement and the effective use of biological fluid, it is desirable to develop biomedical devices which are capable of testing multiple sample inputs at a time.

Attempts to develop biomedical devices which are cheap and capable of testing multiple sample inputs at a time have been disclosed in prior art. U.S. Pat. No. 8,377,710 B2 discloses lateral flow and flow-through sheet-like biomedical devices based on technical innovations involving patterned porous media and a fluid impervious barrier comprising polymerized photoresists. However, since the fluidic flow in U.S. Pat. No. 8,377,710 B2 is lateral (i.e., transported horizontally) and only driven by capillary action, it is found that such lateral fluidic flow is relatively slow and would take a longer duration to reach the test area at the far end along the flow pathway. Further, such slow lateral fluidic flow would generally cause poor fluidic distribution along the flow pathway, and thus the associated amount of fluid reaching the test area would be unequal. Due to such ineffective fluid transport, not only would a relatively large amount of fluid-to-be-assayed be needed for better performance, but also accurate implementation of quantitative analysis would be difficult.

Accordingly, one of the objectives of the present invention is to develop a biomedical device providing effective utilization of biological fluids.

In addition, the pattern in the biomedical devices of U.S. Pat. No. 8,377,710 B2 was formed via using either specific photoresists or wax. Such patterns are vulnerable to organic solvents, such as alcohols, and the flow pathway formed thereby would be destroyed.

In this regard, another objective of this present invention is to develop a biomedical device, coherently providing the excellent resistance against organic solvents and potentially allowing us to perform the organic-solvent-based diagnosis.

### SUMMARY OF THE INVENTION

In view of the aforementioned deficiency in the prior art, in one aspect, the present invention provides a biomedical device comprising a porous hydrophilic substrate; a hydrophobic material; a fluid inlet; and two or more test zones, in which a fluid to be tested vertically flows through the porous hydrophilic substrate and is distributed to the test zones.

In another aspect, the present invention provides a method for making a biomedical device.

The biomedical device of the present invention has at least the following advantages and beneficial effects:

1. The biomedical device of the present invention provides vertical flow pathways and two or more test zones and allows multiple tests to be carried out simultaneously. The testing efficiency and the response time of the biomedical device are improved accordingly.

2. The biomedical device of the present invention effectively enhances the utilization of biological fluids so that tests may be carried out with less biological fluids input or bioassay reagents.

3. The fabrication and assembling of the biomedical device of the present invention are easy. The biomedical device can be fabricated with one single substrate.

### BRIEF DESCRIPTION OF THE DRAWINGS

FIGS. 1(a) and 1(b) are a top view and a bottom view of the biomedical device according to one embodiment of the present invention.

FIGS. 2 to 5 show the results of the flow pathway tests of the biomedical devices according to one embodiment of the present invention.

FIG. 6 shows the results of the organic solvent withstanding test of the biomedical devices according to one embodiment of the present invention.

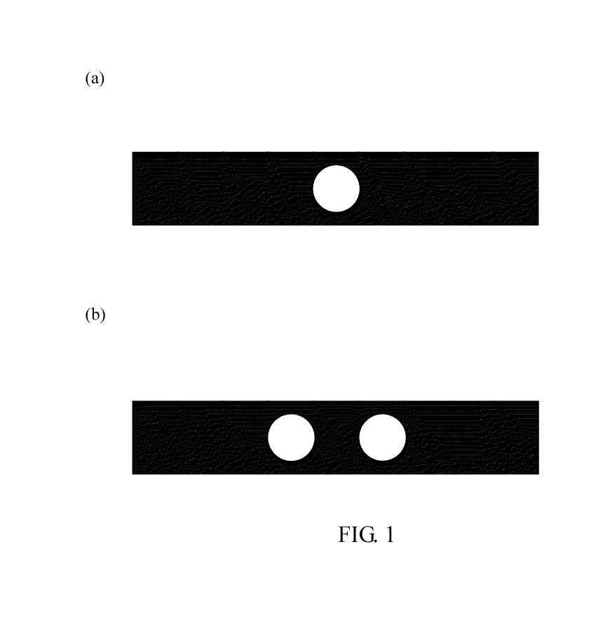

### DETAILED DESCRIPTION OF THE INVENTION

In one embodiment, the present invention is directed to a biomedical device comprising a porous hydrophilic substrate; a hydrophobic material, which is applied to at least part of the porous hydrophilic substrate to form a hydrophobic barrier pattern; a fluid inlet, which is located on the surface of the porous hydrophilic substrate; and two or more test zones, which are positioned on another surface of the porous hydrophilic substrate and comprise bioassay reagents.

The porous hydrophilic substrate of the present invention transports fluids by capillary action. The hydrophobic material is applied to at least part of the porous hydrophilic material to define a pattern of hydrophobic barriers which outlines a channel region within the porous hydrophilic substrate. The channel region further provides flow pathways for the fluid to be assayed and respective fluidic communications between the fluid inlet and the test zones.

The fluid to be assayed flows substantially vertically from the fluid inlet through the pathway, mainly driven by capillary action, surface tension and gravity, and is distributed to the test zones in which assaying reagents are disposed. The assaying reagent tests the fluid and provides a visible color or intensity change for clinical related use.

Compared to the aforementioned prior art lateral fluid transportation, which is driven by capillary action, such substantially vertical fluid transportation of the present invention allows the fluid flow to reach the test zones within a shorter period and improves the fluidic distribution along each flow pathway.

The hydrophilic substrate may include but not be limited to nitrocellulose acetate, cellulose acetate, cellulosic paper, filter paper, tissue paper, writing paper, cloth, porous polymer film and the combination thereof. Selection may be made based on the surface tension of the foregoing materials. A material with suitable surface tension may be used as a hydrophilic substrate to achieve efficient fluid transportation. The hydrophobic material of the present invention has excellent resistance against water and organic solvents, such as alcohols. The hydrophobic material may be a photocuring resin, a thermosetting resin or a thermoplastic resin, preferably a thermosetting acrylate, thermoplastic acrylate, photocuring acrylate, thermosetting silicone resin, thermoplastic silicone resin, photocuring silicone resin, thermosetting fluorocarbon resin, thermoplastic fluorocarbon resin, thermosetting epoxy resin, thermoplastic epoxy resin or photocuring epoxy resin. More preferably, the hydrophobic material is selected from thermoplastic acrylate, photocuring acrylate, thermosetting silicone resin, thermoplastic silicone resin or thermosetting fluorocarbon resin. The hydrophobic material is not reactive with the color reagents or enzyme reagents. Such inert property of the color reagents or enzyme reagents is helpful for maintaining the stability of the color reactions or enzyme reactions that occur in the test zones.

The fluid inlet of the present invention may further contain a hydrophilic gel which does not react with the fluid to improve the fluid absorption rate of the porous hydrophilic substrate. The hydrophilic gel may flow into the fluid pathway and be used as an auxiliary agent for the absorption or immobilization of color reagents or enzyme reagents. The hydrophilic gel useful in the present invention includes, but is not limited to, a natural gel and a synthetic gel. The natural gel may be selected from starch, modified starch, Arabic gum, kink yellow sugarcane gum, karaya gum, tragacanth gum, guar gum, locust bean gum, acacia gum, algin, alginate, carrageenan, polydextrose, hydroxymethyl cellulose, microcrystalline cellulose, carboxymethyl cellulose, pectin, gelatin and casein. The synthetic gel may be selected from polyvinyl tetrahydropyrrolidone, low methoxyl pectin, propylene glycol alginate, hydroxymethyl locust bean gum and hydroxymethyl guar gum.

A biomedical device according to one of the embodiments of the present invention comprises one fluid inlet on one surface of the device and two test zones located on the other surface of the device. The two test zones contain colorless phenolphthalein. When a drop of a basic solution is loaded onto the fluid inlet and absorbed by the porous hydrophilic substrate, within a certain period, the two test zones show a red color resulting from the reaction of the basic solution and phenolphthalein.

A biomedical device according to one of the embodiments of the present invention withstands acetone, alcohol and dimethyl sulfoxide (DMSO) to a certain extent. Such withstanding property shows the present invention's resistance to organic solvents.

The present invention also provides a method for making a biomedical device, comprising:

applying a hydrophobic material to at least part of a porous hydrophilic substrate to form a hydrophobic barrier pattern and outline a fluid inlet on the surface of the porous hydrophilic substrate;

forming two or more test zones on the other surface of the porous hydrophilic substrate; and

disposing bioassay reagents into the test zones.

The cross-sections of the porous hydrophilic substrate to which the hydrophobic material applied may be completely filled with the hydrophobic material, partially filled with the hydrophobic material or excluded from the hydrophobic material. According to one of the preferred embodiments of the present invention, at least part of the cross-sections of the porous hydrophilic substrate to which the hydrophobic material applied are completely filled with the hydrophobic material.

The fluid inlets or test zones of the biomedical device of the present invention are defined by the hydrophobic barrier patterns which are formed by the application of the hydrophobic material. The number of fluid inlets and test zones may be adjusted as needed according to the target test projects such that the biomedical devices of the present invention may carry out one target test, blank test and control test simultaneously or perform multiple target tests at a time.

The process for applying the hydrophobic material to form a hydrophobic barrier pattern and test zones can be any processes well known to a person of ordinary skill in the art. The process includes, but is not limited to, printing, coating or dispensing process. The coating process may be screen printing or inkjet printing. The coating process includes knife coating, roller coating, micro gravure coating, flow coating, dip coating, spray coating, curtain coating or the combination thereof. The application of the hydrophobic material to form a hydrophobic barrier pattern of the present invention is preferably performed by screen printing or inkjet printing.

The biomedical device of the present invention optionally includes a functional substrate. The functional substrate includes, but is not limited to, a natural fiber sheet, glass substrate, silicon substrate or polymeric substrate such as polymethylmethacrylate, polycarbonate or polyethylene terephthalate. The biomedical device of the present invention may include an RFID containing film as a functional substrate to mark test samples. The biomedical device of the present invention may also include a blood cell filter membrane for use in a serum test.

While the foregoing written description of the invention enables one of ordinary skill to make and use what is considered presently to be the embodiments thereof, those of ordinary skill in the art will understand and appreciate the existence of variations, combinations, and equivalents of the specific embodiments described herein. The invention should therefore not be limited by the above described embodiments, but by all embodiments within the scope and spirit of the invention.

### PREFERRED EMBODIMENT OF THE PRESENT INVENTION

### Fabrication of the Biomedical Device, Flow Pathway Test and Solvent Withstanding Test

A hydrophobic material is applied to at least part of a porous hydrophilic substrate by screen printing to form a first hydrophobic barrier pattern, and then is applied to the other surface of the porous hydrophilic substrate by screen printing to form a second hydrophobic barrier pattern. A color reagent is added into the barrier pattern zones for observation.

### The Species of the Hydrophobic Material and the Viscosity Thereof

The species of the hydrophobic materials and their viscosity determined by standard methods are shown in Table 1. The hydrophilic materials A to E are purchased from Eternal Chemical Co., Ltd. (the product models are shown in Table 1).

TABLE 1

| Hydrophobic materials | | | Viscosity(cps) |
|---|---|---|---|
| A | Dow Corning 184 | Thermosetting silicone resin | 6400 |
| B | ETERAD 4650T | Photocuring acrylate | 4400 |
| C | ETERLED GD531 | Thermosetting silicone resin | 3500 |
| D | ETERSOL 15439 | Thermoplastic acrylate | 120 |
| E | ETERLON 4261 | Thermosetting fluorocarbon | 5300 |
| | | resin | |

The hydrophobic materials B, A, D and E are respectively mixed with carbon black to form the hydrophobic materials G, H, I and J.

### B. The Fabrication of the Biomedical Devices

The hydrophobic materials in Table 1 and the porous hydrophilic substrates in Table 2 are used to fabricate the biomedical device of the present invention.

### EXAMPLE 1

### EXAMPLE 1-1

A porous hydrophilic substrate (Waterman #1) is fixed on a screen printing platform. The hydrophobic material D was printed on one surface of the porous hydrophilic substrate by screen printing with a screen having a first surface pattern (single circle pattern) to form a first hydrophobic barrier pattern (single circle pattern). The screen printing operating conditions were as follows:

screen: 100 Mesh Tetron, 20 μm thick emulsion;

squeegee rate for wiping forward: 200 mm/s;

squeegee rate for wiping backward: 365 mm/s; and

spacing between the screen and the screen printing platform: 3.0 mm.

Next, the porous hydrophilic substrate having the first hydrophobic barrier pattern was put in an oven and dried (120° C., 10 to 15 min). The dried porous hydrophilic substrate having the first hydrophobic barrier pattern was turned over and fixed on the screen printing platform, and the hydrophobic material D was printed on the porous hydrophilic substrate by screen printing with a screen having a second surface pattern (double circle pattern) to form a second hydrophobic barrier pattern (double circle pattern). The screen printing operating conditions were as follows:

screen: 100 Mesh Tetron, 20 μm thick emulsion;

squeegee rate for wiping forward: 200 mm/s;

squeegee rate for wiping backward: 365 mm/s; and

spacing between the screen and the screen printing platform 3.0 mm.

Next, the porous hydrophilic substrate having the first and second hydrophobic barrier patterns was put in an oven and dried (120° C., 10 to 15 min) to obtain a biomedical device specimen 1. The diameters of all the circles of the fabricated patterns were 1 cm.

### EXAMPLE 1-2

A porous hydrophilic substrate (Waterman #1) is fixed on a screen priming platform. The hydrophobic material D was printed on one surface of the porous hydrophilic substrate by screen printing with a screen having a first surface pattern (double circle pattern) to form a first hydrophobic barrier pattern (double circle pattern). The screen printing operating conditions were as follows:

screen: 100 Mesh Tetron, 20 μm thick emulsion;

squeegee rate for wiping forward: 200 mm/s;

squeegee rate for wiping backward: 365 mm/s; and

spacing between the screen and the screen printing platform: 3.0 mm.

Next, the porous hydrophilic substrate having the first hydrophobic barrier pattern was put in an oven and dried (120° C., 10 to 15 min). The dried porous hydrophilic substrate having the first hydrophobic barrier pattern was turned over and fixed on the screen printing platform, and the hydrophobic material D was printed on the porous hydrophilic substrate by screen printing with a screen having a second surface pattern (single circle pattern) to form a second hydrophobic barrier pattern (single circle pattern). The screen printing operating conditions were as follows:

screen: 100 Mesh Tetron, 20 μm thick emulsion;

squeegee rate for wiping forward: 200 mm/s;

squeegee rate for wiping backward: 365 mm/s; and

spacing between the screen and the screen printing platform: 3.0 mm.

Next, the porous hydrophilic substrate having the first and second hydrophobic barrier patterns was put in an oven and dried (120° C., 10 to 15 min) to Obtain a biomedical device specimen 1. The diameters of all the circles of the fabricated patterns were 1 cm.

The specimen 1 of the present invention can be fabricated by the method of Example 1-1 or the method of Example 1-2. The difference between these two methods lies in the sequence in which different hydrophobic barrier patterns are applied. The specimens fabricated by these two methods have similar efficacy and performance. Like the fabrication of the specimens in Example 1, in the following Examples 2 to 7, each of biomedical device specimens 2 to7 is fabricated by two different methods, and the efficacy and performance of the specimens fabricated by these various methods are similar.

### EXAMPLE 2

### EXAMPLE 2-1

A porous hydrophilic substrate (Waterman #1) is fixed on a screen printing platform. The hydrophobic material D was printed on one surface of the porous hydrophilic substrate by screen printing with a screen having a first surface pattern (single circle pattern) to form a first hydrophobic barrier pattern (single circle pattern). The screen printing operating conditions were as follows:

screen: 100 Mesh Tetron, 20 μm thick emulsion;

squeegee rate for wiping forward: 200 mm/s;

squeegee rate for wiping backward: 365 mm/s and

spacing between the screen and the screen printing platform: 3.0 mm.

Next, the porous hydrophilic substrate having the first hydrophobic barrier pattern was put in an oven and dried (120° C., 10 to 15 min). The dried porous hydrophilic substrate having the first hydrophobic barrier pattern was turned over and fixed on the screen printing platform, and the hydrophobic material B was printed on the porous hydrophilic substrate by screen printing with a screen having a second surface pattern (double circle pattern) to form a second hydrophobic barrier pattern (double circle pattern). The screen printing operating conditions were as follows:

screen: 100 Mesh Tetron, 20 μm thick emulsion;

squeegee rate for wiping forward: 200 mm/s;

squeegee rate for wiping backward: 365 mm/s; and

spacing between the screen and the screen printing platform: 3.0 mm.

Next, the porous hydrophilic substrate having the first and second hydrophobic barrier patterns was put in an oven and dried (120° C., 10 to 15 min) to obtain a biomedical device specimen 1. The diameters of all the circles of the fabricated patterns were 1 cm.

### EXAMPLE 2-2

A porous hydrophilic substrate (Waterman #1) is fixed on a screen printing platform. The hydrophobic material B was printed on one surface of the porous hydrophilic substrate by screen printing with a screen having a first surface pattern (double circle pattern) to form a first hydrophobic barrier pattern (double circle pattern). The screen printing operating conditions were as follows:

screen: 100 Mesh Tetron, 20 μm thick emulsion;

squeegee rate for wiping forward: 200 mm/s;

squeegee rate for wiping backward: 365 mm/s; and

spacing between the screen and the screen printing platform: 3.0 mm.

Next, the porous hydrophilic substrate having the first hydrophobic barrier pattern was put in an oven and dried (120° C., 10 to 15 min). The dried porous hydrophilic substrate having the first hydrophobic barrier pattern was turned over and fixed on the screen printing platform, and the hydrophobic material D was printed on the porous hydrophilic substrate by screen printing with a screen having a second surface pattern (single circle pattern) to form a second hydrophobic barrier pattern (single circle pattern). The screen printing operating conditions were as follows:

screen: 100 Mesh Tetron, 20 μm thick emulsion;

squeegee rate for wiping forward: 200 mm/s;

squeegee rate for wiping backward: 365 mm/s; and

spacing between the screen and the screen printing platform: 3.0 mm.

Next, the porous hydrophilic substrate having the first and second hydrophobic barrier patterns was put in an oven and dried (120° C., 10 to 15 min) to obtain a biomedical device specimen 1. The diameters of all the circles of the fabricated patterns were 1 cm.

### EXAMPLE 3-1

A biomedical device specimen 3 was fabricated under the conditions of Example 1-1, except that Waterman #4 was used as the porous hydrophilic substrate.

### EXAMPLE 3-2

A biomedical device specimen 3 was fabricated under the conditions of Example 1-2, except that Waterman #4 was used as the porous hydrophilic substrate.

### EXAMPLE 4-1

A biomedical device specimen 4 was fabricated under the conditions of Example 2-1, except that Waterman #4 was used as the porous hydrophilic substrate.

### EXAMPLE 4-2

A biomedical device specimen 4 was fabricated under the conditions of Example 2-2, except that Waterman #4 was used as the porous hydrophilic substrate.

### EXAMPLE 5-1

A biomedical device specimen 5 was fabricated under the conditions of Example 1-1, except that Waterman #40 was used as the porous hydrophilic substrate.

### EXAMPLE 5-2

A biomedical device specimen 5 was fabricated under the conditions of Example 1-2, except that Waterman #40 was used as the porous hydrophilic substrate.

### EXAMPLE 6-1

A biomedical device specimen 6 was fabricated under the conditions of Example 2-2, except that Waterman #40 was used as the porous hydrophilic substrate.

### EXAMPLE 6-2

A biomedical device specimen 6 was fabricated under the conditions of Example 1-2, except that Waterman #40 was used as the porous hydrophilic substrate.

### EXAMPLE 6-3

A biomedical device specimen 7 was fabricated under the conditions of Example 6-2, except that a hydrophilic material G was used as the hydrophobic material for forming the first hydrophobic barrier pattern (double circle pattern), and the hydrophobic material G was a composition of the hydrophobic material B and carbon black dispersed in the hydrophobic material B.

### EXAMPLE 7-1

A biomedical device specimen 8 was fabricated under the conditions of Example 1-1, except that a hydrophilic material H was used as the hydrophobic material for forming the second hydrophobic barrier pattern (doable circle pattern), and the hydrophobic material H was a composition of the hydrophobic material A and carbon black dispersed in the hydrophobic material A.

### EXAMPLE 8-1

A biomedical device specimen 9 was fabricated under the conditions of Example 3-1, except that a hydrophilic material D was used as the hydrophobic material for forming the second hydrophobic barrier pattern (double circle pattern), and the hydrophobic material 1 was a composition of the hydrophobic material D and carbon black dispersed in the hydrophobic material D.

### EXAMPLE 9-1

A biomedical device specimen 10 was fabricated under the conditions of Example 5-1, except that a hydrophilic material D was used as the hydrophobic material for forming the second hydrophobic barrier pattern (double circle pattern), and the hydrophobic material J was a composition of the hydrophobic material E and carbon black dispersed in the hydrophobic material E.

The results of the foregoing examples are summarized in Table 3 below.

| | Hydrophobic | Hydrophobic | | |
|---|---|---|---|---|
| | material | material | Porous | |
| | for forming a | for forming a | hydro- | Bio- |
| | hydrophobic | hydrophobic | philic | medical |
| | barrier pattern | barrier pattern | substrate | device |
| Exam- | having single | having double | Waterman | spec- |
| ples | circle pattern | circle pattern | # | imens |
| 1-1 | D | D | 1 | 1 |
| 1-2 | D | D | 1 | 1 |
| 2-1 | D | B | 1 | 2 |
| 2-2 | D | B | 1 | 2 |
| 3-1 | D | D | 4 | 3 |
| 3-2 | D | D | 4 | 3 |
| 4-1 | D | B | 4 | 4 |
| 4-2 | D | B | 4 | 4 |
| 5-1 | D | D | 40 | 5 |
| 5-2 | D | D | 40 | 5 |
| 6-1 | D | B | 40 | 6 |
| 6-2 | D | B | 40 | 6 |
| 6-3 | D | G | 40 | 7 |
| 7-1 | D | H | 1 | 8 |
| 8-1 | D | I | 4 | 9 |
| 9-1 | D | J | 40 | 10 |

### C. Biomedical Device Flow Pathway Test

1. Test of Introducing Reagents into the Single Circle Pattern and Observing the Color Changes in the Double Circle Pattern Zones (Tests 1-10)

### Test 1

A starch solution was applied to each of the double circle patterns of the biomedical device specimen 1 by using a cotton swab, and then the biomedical device specimen 1 was fixed by pasting a transparent tape (3M). Next, 20 μL iodine tincture was dropped into the single circle pattern of the biomedical device specimen, and observations were made of any significant color change that occurred in the double circle patterns where the starch solution had been applied.

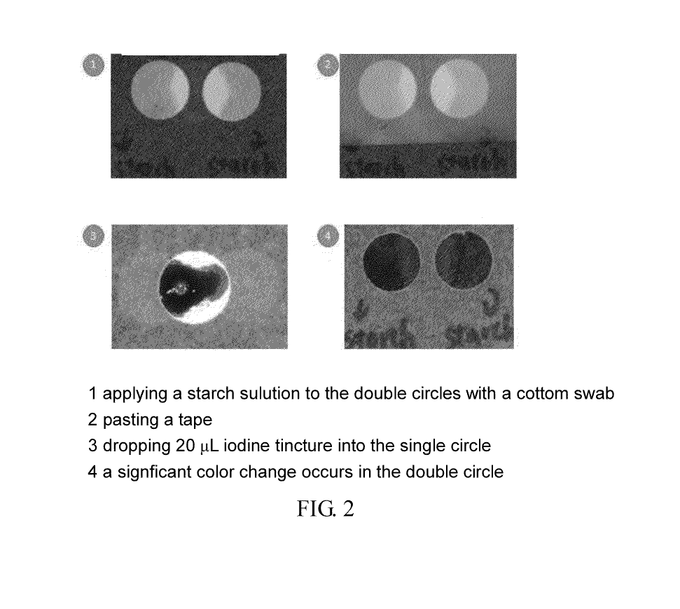

According to the observation results shown in FIG. 2, a significant color change occurred in the double circle patterns where the starch solution had been applied. Such result indicates that an interconnected flow pathway structure exists between the single circle and each circle of the double circle patterns.

### Test 2

A starch solution was applied to one of the double circle patterns of the biomedical device specimen 1 by using a cotton swab, and then the biomedical device specimen 1 was fixed by pasting a transparent tape (3M). Next, 20 μL iodine tincture was dropped into the single circle pattern of the biomedical device specimen, and observations were made of any significant color change that occurred in the double circle pattern where the starch solution had been applied.

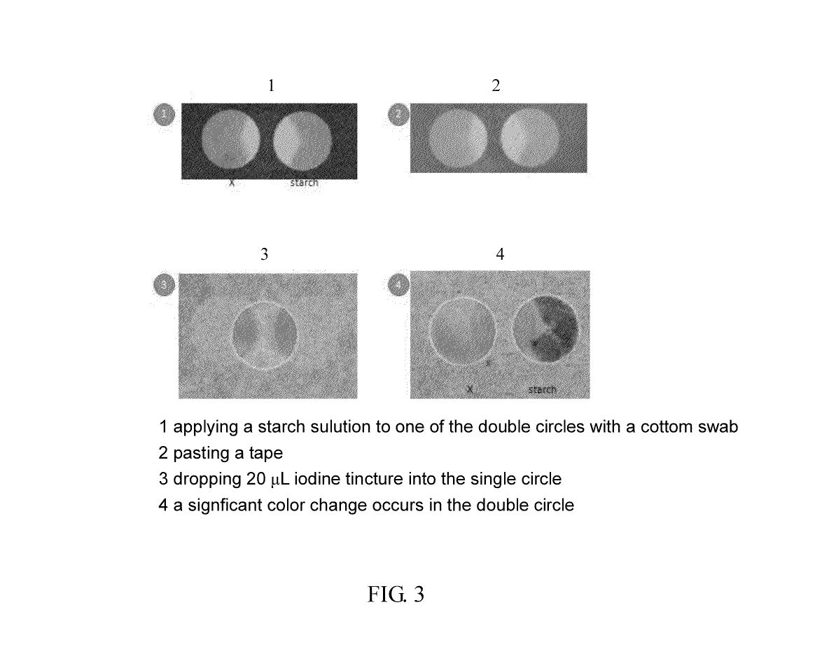

According to the observation results shown in FIG. 3, in the double circle patterns, significant color change occurred in the circle where the starch solution was applied, while no color change was observed in the one to which the starch solution was not applied. Such results indicate that an interconnected flow pathway structure exists between the single circle pattern and each circle of the double circle patterns.

### Test 3

A nitrite indicator (purchased from Merck) was applied to each circle of the double circle patterns of the biomedical device specimen 1 by using a cotton swab, and then the biomedical device specimen 1 was fixed by pasting a transparent tape (3M). Next, 20 μL nitrite solution (NO_{2 (aq)}⁻ reagent, nitrite test, purchased from Merck) was dropped into the single circle pattern of the biomedical device specimen, and observations were made of any significant color change that occurred in the double circle patterns where the nitrite indicator had been applied.

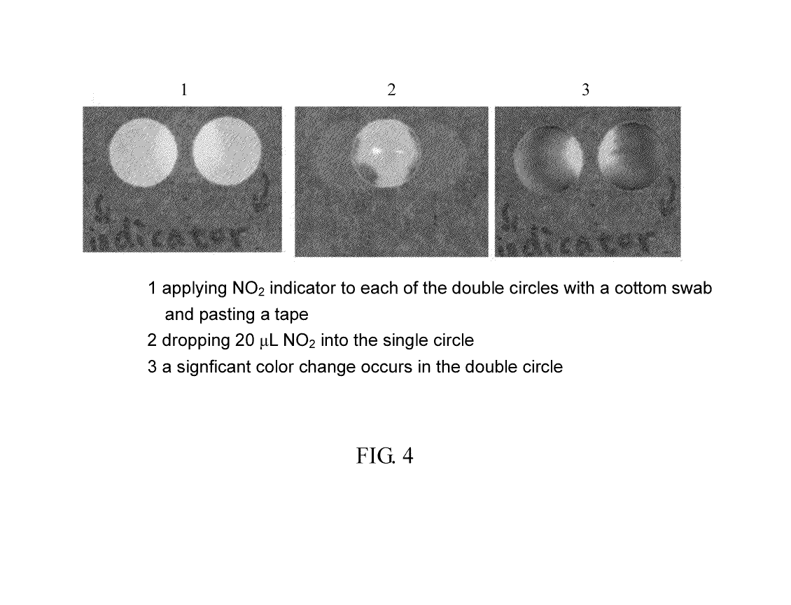

According to the observation results shown in FIG. 4, a significant color change from original colorless to pink occurred in the double circle patterns where the nitrite indicator was applied. Such results indicate that an interconnected flow pathway structure exists between the single circle pattern and each circle of the double circle patterns.

### Test 4

A nitrite indicator (purchased from Merck) was applied to one of the double circle patterns of the biomedical device specimen 1 by using a cotton swab, and then the biomedical device specimen 1was fixed by pasting a transparent tape (3M). Next, 20 μL nitrite solution (NO_{2 (aq)}⁻ reagent, nitrite test, purchased from Merck) was dropped into the single circle pattern of the biomedical device specimen, and observations were made of any significant color change that occurred in the double circle pattern where the nitrite indicator had been applied.

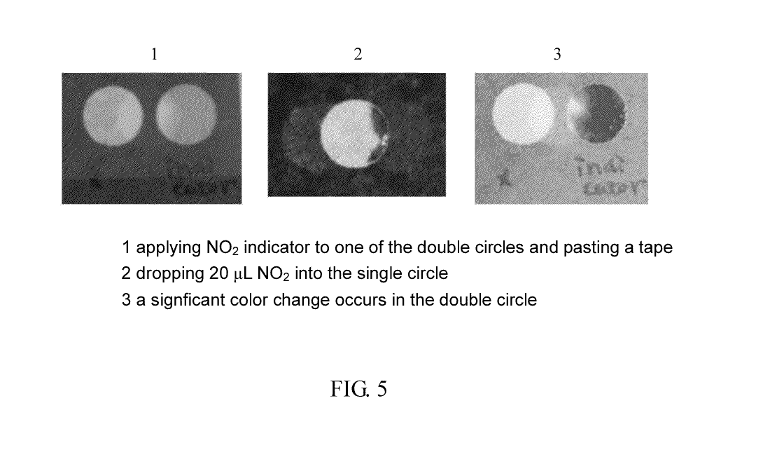

According to the observation results shown in FIG. 5, in the double circle patterns, a significant color change occurred in the circle where the nitrite indicator was applied, while no color change occurred in the one to which the nitrite indicator was not applied. Such results indicate that an interconnected flow pathway structure exists between the single circle pattern and each circle of the double circle patterns.

### Test 5

A universal indicator (purchased from Merck) was applied to each circle of the double circle patterns of the biomedical device specimen 1 by using a cotton swab. Next, 20 μL NaOH (5%) was dropped into the single circle pattern of the biomedical device specimen, and observations were made of any significant color change that occurred in the double circle patterns where the universal indicator had been applied.

It is found from the observation results that a significant color change occurred in the double circle patterns where the universal indicator was applied. The color changed from original yellow green to blue purple. Such results indicate that an interconnected flow pathway structure exists between the single circle pattern and each circle of the double circle patterns.

### Test 6

A universal indicator (purchased from Merck) was applied to one of the double circle patterns of the biomedical device specimen 1 by using a cotton swab. Next, 20 μL NaOH (5%) was dropped into the single circle pattern of the biomedical device specimen, and observations were made of any significant color change that occurred in the double circle pattern where the universal indicator had been applied.

It is found from the observation results that in the double circle patterns, a significant color change occurred in the circle where the universal reagent was applied; while no color change occurred in the one without to which the nitrite indicator was not applied. Such results indicate that an interconnected flow pathway structure exists between the single circle pattern and each circle of the double circle patterns.

### Test 7

A universal indicator (purchased from Merck) was applied to each of the double circle patterns of the biomedical device specimen 1by using a cotton swab. Next, 20 μL HCl (5%) was dropped into the single circle pattern of the biomedical device specimen, and observations were made of any significant color change that occurred in the double circle patterns where the universal indicator had been applied.

It is found from the observation results that a significant color change occurred in the double circle patterns where the universal reagent was applied. The color changed from original yellow green to light orange. Such results indicate that an interconnected flow pathway structure exists between the single circle pattern and each circle of the double circle patterns.

### Test 8

A universal indicator (purchased from Merck) was applied to one of the double circle patterns of the biomedical device specimen 1 by using a cotton swab. Next, 20 μL HCl (5%) was dropped into the single circle pattern of the biomedical device specimen, and observations were made of any significant color change that occurred in the double circle pattern where the universal indicator had been applied.

It is found from the observation results that in the double circle patterns, a significant color change occurred in the circle where the universal reagent was applied; while no color change occurred in the one to which the nitrite indicator was not applied. Such results indicate that an interconnected flow pathway structure exists between the single circle pattern and each circle of the double circle patterns.

### Test 9

To estimate the diffusion performance of different porous hydrophilic substrates, Test 4 was performed on biomedical device specimens 1, 3 and 5 under the same conditions. Nitrite indicators were dropped into the single circle pattern of the biomedical device specimens, and then the duration from the instant the color started to change to the instant that the color spread to the entire single circle pattern was measured.

It is observed that the flow diffusion in specimen 1 completed within a shorter time than taken in specimen 3 or 5, and the diffusion in specimen 3 completed within a shorter time than taken in specimen 5. These results are due to the pore size differences among the porous hydrophilic substrates of specimens 1, 3 and 5. That is, among specimens 1, 3 and 5, the porous hydrophilic substrate of specimen 1, which has the greatest pore diameter, diffuses the fastest, and the porous hydrophilic substrate of specimen 5, which has the smallest pore diameter, diffuses the slowest.

### Test 10

To estimate the diffusion performance of different porous hydrophilic substrates, Test 2 was performed on biomedical device specimens 2, 4 and 6 under the same conditions. Nitrite indicators were dropped into the single circle pattern of the biomedical device specimens, and then the duration from the instant the color started to change to the instant that the color spread to the entire single circle pattern was measured.

It is observed that the flow diffusion in specimen 2 completed within a shorter time than taken in specimen 4 or 6, and the diffusion in specimen 4 completed within a shorter time than taken in specimen 6. Such results are due to the pore size differences among the porous hydrophilic substrates of specimens 2, 4 and 6. That is, among specimens 2, 4 and 6, the porous hydrophilic substrate of specimen 2, which has the greatest pore diameter, diffuses the fastest, and the porous hydrophilic substrate of specimen 6, which has the smallest pore diameter, diffuses the slowest.

### Solvent Withstanding Test

### Test 11

An acetone/red ink mixture, an ethanol/red ink mixture and a DMSO/red ink mixture were respectively dropped into the single circle pattern of three biomedical device specimens 7 fabricated in the foregoing Example 6-3, and then the diffusion of the red ink in the presence of an organic solvent at different time points (5 sec, 10 sec, 15 sec, 20 sec and 25 sec) was observed.

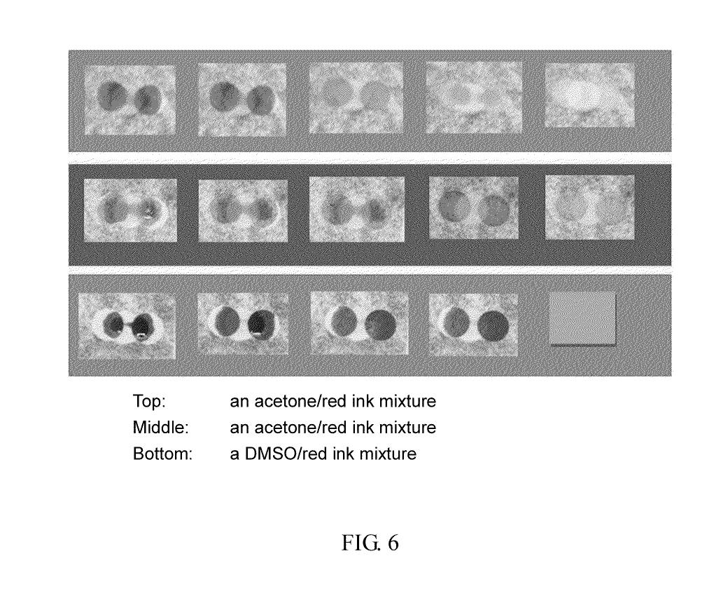

The observation results are shown in FIG. 6. The double circle patterns of the three specimens are intact without any side etching. The tests were carried out under the same conditions except for specimen 7 being replaced by specimens 8-10. Similar results were observed. Such results indicate not only that the biomedical devices of the present invention provide interconnected flow pathway between the single circle pattern and the double circle pattern but also that all the hydrophobic barrier patterns formed by different hydrophilic materials (A-E and carbon-black-containing G-J) of the present invention have excellent organic solvent resistance.

What is claimed is:

1. A biomedical device comprising
a porous hydrophilic substrate;
a hydrophobic material,
wherein the hydrophobic material is applied to at least part of the porous hydrophilic substrate to form a hydrophobic barrier pattern;
a fluid inlet, which is located on the surface of the porous hydrophilic substrate;
two or more test zones, which are positioned on another surface of the porous hydrophilic substrate and comprise bioassay reagents;
wherein the fluid vertically flows through the porous hydrophilic substrate and is distributed to said test zones.

2. The biomedical device of claim 1, wherein the hydrophobic material is a photocuring resin, thermosetting resin or thermoplastic resin.

3. The biomedical device of claim 1, wherein the hydrophobic material comprises a thermoplastic acrylate, photocuring acrylate, thermosetting silicone resin, thermoplastic epoxy resin or thermosetting epoxy resin.

4. The biomedical device of claim 1, wherein the hydrophobic material has resistance against water and organic solvents.

5. The biomedical device of claim 1, wherein the fluid inlet is defined by the hydrophobic barrier pattern on the surface of the porous hydrophilic substrate.

6. The biomedical device of claim 1, wherein the two or more test zones are defined by the hydrophobic barrier pattern on another surface of the porous hydrophilic substrate.

7. The biomedical device of claim 1, wherein the hydrophilic substrate is selected from a group comprising nitrocellulose acetate, cellulose acetate, cellulosic paper, filter paper, tissue paper, writing paper, cloth, porous polymer films and the combination thereof.

8. The biomedical device of claim 1, wherein the fluid inlet further comprises a hydrophilic paste.

9. A method for making a biomedical device, comprising:
applying a hydrophobic material to at least part of a porous hydrophilic substrate to form a hydrophobic barrier pattern and outline a fluid inlet on the surface of the porous hydrophilic substrate;
forming two or more test zones on the other surface of the porous hydrophilic substrate; and
disposing bioassay reagents into the test zones.

10. The method according to claim 9, wherein the application of a hydrophobic material may be carried out by screen printing or inkjet printing.
